(19) United States
(12) Patent Application Publication
(10) Pub. No.: US 2018/0071418 A1
(43) Pub. Date: Mar. 15, 2018

(54) PROCESS CHALLENGE DEVICE FOR AUTOMATED ENDOSCOPE REPROCESSOR

(71) Applicant: 3M INNOVATIVE PROPERTIES COMPANY, St. Paul, MN (US)

(72) Inventor: G. MARCO BOMMARITO, STILLWATER, MN (US)

(21) Appl. No.: 15/564,447

(22) PCT Filed: Apr. 5, 2016
(86) PCT No.: PCT/US2016/025970

§ 371 Date: Oct. 5, 2017

Related U.S. Application Data
(60) Provisional application No. 62/145,323, filed on Apr. 9, 2015.

(51) Int. Cl. A61L 2/18; A61L 2/28; A61B 1/12

(52) U.S. Cl. CPC A61L 2/18; A61L 2/28; A61L 2/24; A61B 1/125; A61B 1/122

(57) ABSTRACT

The present disclosure describes a novel monitoring system which enables a user to verify the effectiveness of the disinfection cycle provided by an automated endoscope reprocessor (AER). The disclosure proposes the use of chemical and/or biological indicators integrated within a process challenge device that mimics the challenge posed by an endoscope processed in the AER.

### CROSS REFERENCE TO RELATED APPLICATIONS

This application claims priority to U.S. Provisional Patent Application No. 62/145,323, filed Apr. 9, 2015, the disclosure of which is incorporated by reference in its entirety herein.

### BACKGROUND

Endoscopy procedures play a beneficial role in the prevention, diagnosis and treatment of disease. Endoscopy procedures are performed using complex, reusable, flexible instruments that, when inserted into the body, may become heavily contaminated with patient biomaterial and microorganisms, including potential pathogens. Careful reprocessing of flexible endoscopes between patients is critical to reducing the risk of cross-contamination and the possible transmission of pathogens.

Flexible endoscopes are rated as semi-critical according to the Spaulding classification for medical devices and therefore it is required that these devices be decontaminated by high-level disinfection. Thus, it is recommended that both endoscopes and reusable accessories be frequently visually inspected in the course of their use and reprocessing, including before, during and after use, as well as after cleaning and before high-level disinfection. However, a visually based method of verification has severe limitations when applied to flexible endoscopes because the complex, narrow lumens in these devices cannot be directly visually inspected.

Automated endoscope reprocessors (AERs) are used to clean and disinfect flexible endoscopes to a level that mitigates transmission of pathogenic organisms and disease between patients who are subject to an endoscopic procedure. Typically, the only information available to a user is the parametric information provided by the AER equipment itself which consists primarily of time and temperature information. The AER does not monitor chemical parameters capable of establishing the effectiveness of the disinfection cycle.

Existing chemical or biological indicators for use with AER's do not take into account the challenge introduced by long narrow lumens that provide an environment wherein microorganisms are difficult to remove and can potentially colonize the entire endoscope.

### SUMMARY

In an embodiment, a process challenge device for a liquid disinfecting step is described, wherein the device comprises: a liquid inlet and a liquid outlet, said inlet and outlet connected by a channel, wherein said channel is designed in a tortuous path to mimic the geometry of an endoscope, and at least one indicator positioned within the channel.

In a further embodiment, a method for determining the quality of disinfection in an AER is described, wherein the method comprises:
a. Providing within the AER a challenge device comprising:
   i. a liquid inlet and a liquid outlet, said inlet and outlet connected by a channel, wherein said channel is designed in a tortuous path to mimic the geometry of an endoscope,
   ii. at least one indicator positioned within the channel
b. Analyzing the indicator to confirm whether desired process conditions have been met.

### BRIEF DESCRIPTION OF THE DRAWINGS

FIG. 1 is a top view of an indicator device in one embodiment of the disclosure.

FIG. 2 is a top view of an indicator device in a further embodiment of the disclosure.

FIG. 3 is a cross-sectional view of the device of FIG. 2 taken along line 3-3

### DETAILED DESCRIPTION

The present disclosure describes a novel monitoring system which enables a user to verify the effectiveness of the disinfection cycle provided by an automated endoscope reprocessor (AER). The disclosure proposes the use of chemical and/or biological indicators integrated within a process challenge device that mimics the challenge posed by an endoscope processed in the AER.

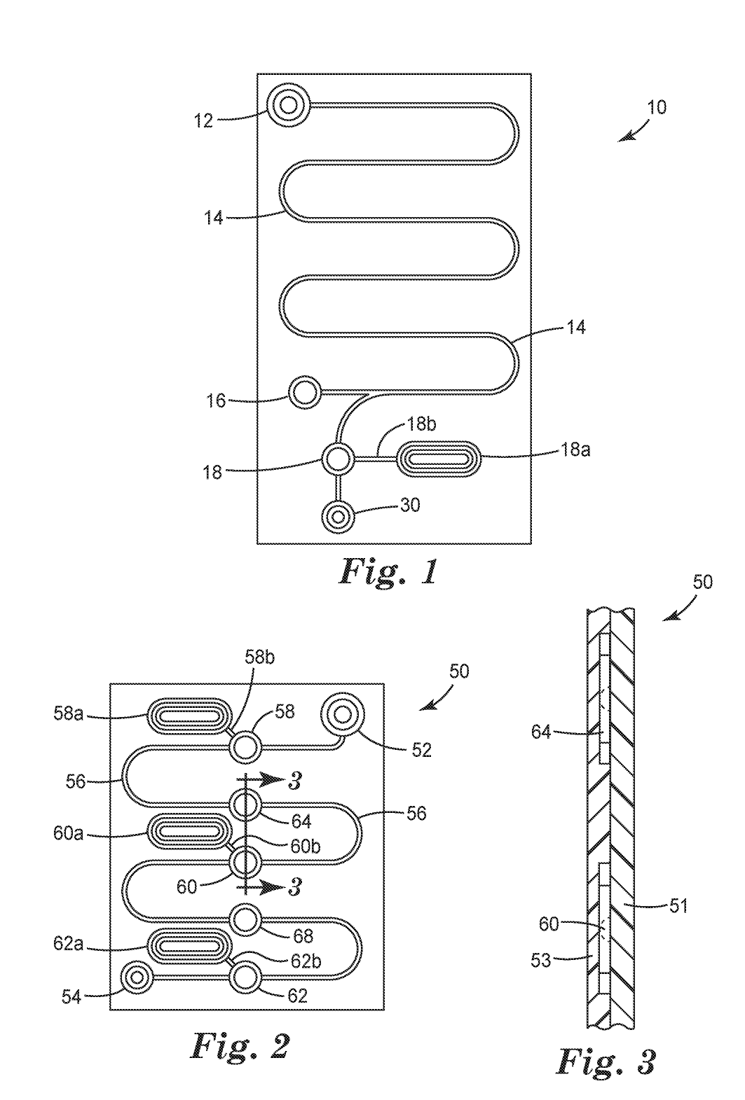

FIG. 1 shows a first embodiment of an exemplary indicator device 10 having an AER connection port 12 at one end, wherein the connection port 12 is fluidly connected to a microfluidic channel 14 which extends along an arcuate path within the indicator device 10. The channel 14 is further in fluid communication with one or more indicator devices 16, 18 along the arcuate path before leading to an exit opening 30. In the exemplary embodiment of FIG. 1, the indicator device 16 is a chemical indicator and the indicator device 18 is a biological indicator, wherein the biological indicator 18 is further in fluid communication with growth media contained within a frangible growth media capsule 18a. The pathway 18b between the biological indicator 18 and growth media capsule 18a provides a conduit for fluid communication between the biological indicator 18 and growth media once a frangible member of the growth media capsule 18a is ruptured.

FIG. 2 shows a further embodiment of the present disclosure wherein indicator device 50 is configured with a fluid inlet 52 and fluid outlet 54 connected by an arcuate microfluidic channel 56. Along the length of the channel 56, numerous chemical 64, 68 and biological indicators 58, 60, 62 are displaced. Each of the biological indicators 58, 60, 62, are coupled with corresponding growth media capsules 58a, 60a, 62a, with pathways 58b, 60b, 62b providing fluid communication with the biological indicators 58, 60, 62 once a frangible member of the growth media capsules 58a, 60a, 62a are fractured.

FIG. 3 shows a cross section of the indicator device 50 of FIG. 2 taken along line 3-3, showing the device 50 composed of two layers of material 51, 53. Chemical indicator 64 and biological indicator 60 are disposed in layer 53 and the microfluidic channel may be disposed in layer 51 (not shown). Alternatively, both the indicators and microfluidic channel may be disposed in a single layer of material.

As described above, the indicator devices 10, 50 include at least one chemical and/or biological indicator on a single device which also includes a microfluidic channel to simulate the load or challenge posed to an automated endoscope reprocessor (AER) by a flexible endoscope. The connection port 12, 52 at one end of the microfluidic channel 14, 56 allows attachment of the device 10, 50 directly to the AER using an appropriate harness. In an embodiment, the device contains test chambers holding a chemical indicator to monitor the minimum effective concentration (MEC) of the disinfectant, and a biological indicator capable of quantifying the log reduction in viable microorganisms achieved during the disinfection cycle. The microfluidic channel 14, 56 is open ended to allow for continuous flow of disinfectant through the device 10, 50 over the entire cycle.

In use a user would first connect the device 10, 50 directly to the AER machine using a harness modified from that used to connect an endoscope to allow connection of the device 10, 50 in parallel to the endoscope. The device 10, 50 would be placed in the basin of the AER that also holds the scope to be reprocessed and would be fully immersed in disinfectant during the cycle. After completion of the cycle, the user would disconnect the device 10, 50 from the AER and first visualize the colorimetric response of the chemical indicator to establish if the MEC was achieved. If the biological indicator was based on detecting a response from the growth of viable organisms coated directly in the chamber of the device or on a suitable substrate placed in the chamber of the device, the user would next activate the biological indicator by breaking a frangible vial containing growth media allowing media to enter the chamber holding the indicator. The device would then be placed in an incubator also capable

The arcuate path of the microfluidic channel is designed to mimic a full length flexible endoscope on the basis of Poiseuille's law. In the case of laminar flow, the volume flowrate is given by the pressure difference divided by the viscous resistance. This resistance depends linearly upon the viscosity and the length, but the fourth power dependence upon the radius is dramatically different. Poiseuille's law is found to be in reasonable agreement with experiment for uniform liquids (Newtonian fluids) in cases where there is no appreciable turbulence.

According to Poiseuille's law, the volumetric flowrate is given by:

$$Volumetric Flowrate = = \frac{{P}_{1} - {P}_{2}}{} = \frac{π ( {P}_{1} - {P}_{2} ) {r}^{4}}{}$$

Where the resistance to flow [symbol] is given by:
Where η is the viscosity of the liquid.

$$= \frac{B η L}{π {r}^{4}}$$

This advantageously allows mimicking the challenge posed to an AER by a flexible endoscope using a considerably condensed format. For example, some of the larger gastrointestinal flexible endoscopes have 2 m long lumens 5 mm in diameter. Given a disinfectant with a known viscosity η, the resistance to flow [symbol] will be proportional to L/r⁴, which for the example is equal to 51.2 mm⁻³. To simulate an equivalent resistance using a microfluidic channel 1 mm in diameter, the length L necessary would be only 3.2 mm.

Suitable chemical indicators for use with the devices described herein would comprise a colorimetric system to verify the minimum effective concentration (MEC) of disinfectant liquid. One possible system would be based on the reaction of a commonly used high level disinfectant, ortho-phthalaldehyde with sodium sulfite disposed on a substrate. The reaction forms a sulfite addition product and an equivalent amount of base according to the following reaction:

C₆H₄(CHO)₂+2Na₂SO_{g}+2H₂O→C₆H₄(CH(SO_{g}Na)OH)₂+2NaOH

If sufficient ortho-phthalaldehyde is present, the increase in pH causes a color change in the pH indicator also disposed on the substrate. When the concentration of ortho-phthalaldehyde is sufficient, the local pH typically rises above 11 and a color change to a deep purple occurs. There are several suitable pH dyes that can be used in this indication. A similar reaction scheme can be used to test MEC for glutaraldehyde (GA) disinfectants, another common class of HLD (High Level Disinfection) chemicals used in reprocessing flexible endoscopes. The chemical indication could be also configured to be an integrator, meaning that it will measure not just whether the disinfectant is above a certain concentration but for how long it was at that concentration. This could be done by providing an indicator system where the colorimetric response was proportional to a dosage or contact time. For example, by disposing the indicator system along a wicking strip rather than in a dot, and allowing for capillary action in the wicking material to dictate the flow of disinfectant along the strip, visualization of the colorimetric front along the strip would then become an indication of time as well as MEC. The porosity of the strip would be chosen to achieve to desired movement of disinfectant along the strip for a given cycle duration. The wicking strip could be made of an appropriate membrane or filtration material but it could also be engineered as an additional microfluidic component that forms a monolithic structure along with the challenge channel of the device.

The biological indicator should be capable of verifying the disinfection efficacy of the cycle. It could work in a manner analogous to current biological indicators designed to monitor various sterilization modalities. As such, it should be based on using a biological entity that can be quantified with respect to its biological viability. It may be possible to use spores or weakened/injured spores as the biological indicator. The primary advantage of using spores in this application is that they are “shelf stable” for long times at room temperature. Germination and growth of the spores is not easily triggered except by design. In this application it may be possible to simply measure the amount of viable spores present after a disinfection cycle in the AER and compare it to the predetermined amount of spores placed in the chamber of the device. That difference in the spore population pre and post disinfection could then be compared to an expected difference for an effective cycle, and within a certain tolerance window, a determination could be made on whether the disinfection cycle was effective or not (pass or fail). The measured difference would also quantify the log reduction achieved during the cycle. If spores were found to be too resistant to be affected by the disinfectant used in AERs, another potential biological entity useful in this indication could be an appropriate yeast. For example, Saccharomyces cerevisiae is a species of yeast that could be employed in this concept. It is a yeast cell instrumental to winemaking, baking, and brewing and it is one of the most intensively studied eukaryotic model organisms in molecular and cell biology. Rapid detection of the biological indication could be achieved using a florescence based enzymatic reaction. Glucosidase assays using fluorogenic substrates are one such class. For example, β-Glucosidase catalyzes the breakdown of the β-glucosidic linkage in the fluorogenic substrate, β-MUG, to release its component moieties glucose and the fluorescent compound 4-MU. The activity of this enzyme can then be measured as an increase in fluorescence over time from germinated spore suspensions. The reaction is potentially quantitative and could be used to determine the difference from a predetermined initial spore population prior to the initiation of a disinfection cycle to a final spore population upon completion of the disinfection cycle. Another means of determining the efficacy of the disinfection cycle may be to measure the kinetics of the increasing fluorescence signal from the viable spores remaining after disinfection. The pass/fail determination may then be based on how quickly the fluorescent intensity reached a given level. It would also be possible to use colorimetric assays instead of fluorescence based assays, although one would expect these to be less sensitive. It may also be possible for the enzymatic assay to drive an electrochemical response. In this mode rather than integrating light signals, one would either measure changes in potential (coulometric) or current flow (amperometric).

In addition to the embodiments described above, other form factors may be contemplated for the application taught in the current disclosure. For example, multiple channel lengths could be built on a single card to mimic different types of endoscopes.

Also, as described above, the device could have multiple biological and chemical indicators disposed within the channel path to indicate multiple challenges simultaneously. This would be useful if a user wished to have a single device apply to a variety of scope designs (lumen lengths and diameters).

In other embodiments, the device could be designed so that the microfluidic channel also included dead volumes either above or below the plane of flow as well as within that plane, to simulate valves and other dead flow ends common to the design of many flexible endoscopes. Indicators could be disposed at these locations to verify that an appropriate cycle was completed.

In addition to chemical and biological responses the indicator could also be created to monitor physical parameters of the disinfection cycle such as time and temperature. For example a time-temperature indicator in analogy to a 3M Sterigage or a 3M Monitor Mark indicator could be included to measure independently from the AER instrumentation the integrated time-temperature profile of the disinfection cycle. The time-temperature indicator would be designed to have a threshold temperature above which the indicating material flows by wicking along a strip of a filtration material or an engineered microfluidic element. The indicating material's rheology would be chosen to have a temperature dependent viscosity or viscoelastic response to match the activation energy describing the time-temperature profile of the disinfection cycle. The wicking element would have a porosity chosen to dictate a given amount of travel for a given viscosity of the indicating fluid.

In a further example, rather than using a generally planar device having a channel as the challenge device, the endoscope itself could provide the challenge. In this configuration combination biological and chemical flow-through indicators could be placed upstream and/or downstream of the flexible endoscope and read after completion of the cycle in a manner analogous to that described above for the device.

It may be also possible to create a set of biological and chemical indicators that mount to the valve openings in the control head of the endoscope instead of the typical “sled” that is used when the endoscope is place in the AER.

Finally, it may also be possible to have “macroscopic” challenge devices where an identical length of tubing with the same diameter as the endoscope being disinfected is wound around a spool with a flow through combo biological/chemical indicator attached at the distal end of the monitoring device.

1. A process challenge device for a liquid disinfecting step comprising:
(a) a liquid inlet and a liquid outlet, said inlet and outlet connected by a channel, wherein said channel is designed in a tortuous path to mimic the geometry of an endoscope,
(b) at least one indicator positioned within the channel.

2. The device of claim 1, wherein the channel has a primary path and one or more secondary paths.

3. The device of claim 2, wherein the at least one indicator is positioned along a secondary path.

4. The device of claim 1, wherein the device contains at least one chemical indicator and at least one biological indicator.

5. The device of claim 1, wherein the device is generally planar.

6. A method for determining the quality of disinfection in an AER, the method comprising:
a. providing within the AER a challenge device comprising:
i. a liquid inlet and a liquid outlet, said inlet and outlet connected by a channel, wherein said channel is designed in a tortuous path to mimic the geometry of an endoscope,
ii. at least one indicator positioned within the channel, and
b. analyzing the indicator to confirm whether desired process conditions have been met.

7. The method of claim 6, wherein the channel has a primary path and one or more secondary paths.

8. The method of claim 7, wherein the at least one indicator is positioned along a secondary path.

9. The method of claim 6, wherein the device contains at least one chemical indicator and at least one biological indicator.

10. The method of claim 6, wherein the device is generally planar.
